(19) United States
(12) Patent Application Publication
(10) Pub. No.: US 2015/0107338 A1
(43) Pub. Date: Apr. 23, 2015

(54) Pressure Measurement Device and Method

(71) Applicant: Ronald Billett, San Jose, CA (US)

(72) Inventor: Ronald Billett, San Jose, CA (US)

(21) Appl. No.: 14/583,735

(22) Filed: Dec. 28, 2014

(51) Int. Cl. G01N 7/00; A61B 3/16; G01L 9/00

(52) U.S. Cl. CPC G01N 7/00; G01L 9/008; A61B 3/16
USPC 73/31.04

(57) ABSTRACT

Disclosed is a device and method for measuring pressure inside a deformable vessel containing a compressible gas, by the transfer of pressure inside the vessel to a primarily rigid chamber external to the vessel through a flexible membrane covering a window in the chamber, which is filled with a suitable incompressible fluid. The pressure transfer is effectuated by either pressing the vessel against the chamber or the reverse. The transferred pressure is then measured by a traditional gauge or other available pressure sensor. Depending on the elasticity of the vessel exterior wall, a fixed area for compressing a flat area of the vessel exterior wall and transfer membrane is chosen. At this chosen amount of deformation, the pressure differential between the vessel interior and the exterior chamber is zero, facilitating the external measurement of the vessel internal gas pressure.

### FIELD OF THE INVENTION

The present invention is generally related to the field of pressure measurement.

### BACKGROUND OF THE INVENTION

Pressure measurement has a history which begins in the seventeenth century Italian Renaissance with the invention of the barometer. The contemporaneous understanding of atmospheric air pressure fundamentally changed the mechanical understanding of how air exerts pressure against the human body and vessels. In the following centuries, a variety of mechanical and electrical devices would be used to measure pressure, from the first mechanical barometer, the aneroid barometer invented in 1843, to piezoresistive silicon sensors used in some modern smart phones.

One particular pressure measurement application remains problematic, namely measuring pressure inside a sealed deformable vessel. Two longstanding specific applications of this type include the tennis ball and the human eye.

In the case of a tennis ball, since the balls have no valve, traditional tools for measuring the internal pressure of the ball are not available. The official International Tennis Federation rules specify that the height of a ball's bounce should be used as the means to measure the ball's internal pressure.

Pressure inside the human eye is known as intraocular pressure, and is normally regulated by fluid passed through the nasolacrimal duct. When the duct is blocked, intraocular pressure may rise, one of the causes of glaucoma. Various methods and devices have been developed, including variations of the applanation tonometer. Due to the deformation sensitivity of tonometer devices, they are generally pressed directly against the eyeball. Such devices are difficult or impossible to operate through the eyelid, and thus must be kept sterile and the eye (sclera) must be anesthetized.

### SUMMARY OF THE INVENTION

Disclosed is a method for measuring pressure inside a deformable vessel containing a compressible gas, by the transfer of pressure inside the vessel to a primarily rigid chamber external to the vessel through a flexible membrane covering a window in the chamber, which is filled with a suitable incompressible fluid, removing all air. The pressure transfer is effectuated by either pressing the vessel against the chamber or the reverse. The transferred pressure is then measured by a traditional gauge or other available pressure sensor. Depending on the elasticity of the vessel exterior wall, a fixed area for compressing a flat area of the vessel exterior wall and transfer membrane is chosen. At this chosen amount of deformation, the pressure differential between the vessel interior and the exterior chamber is zero, facilitating the external measurement of the vessel internal pressure.

In various embodiments, the chamber pressure measurement is calibrated so that when the deformable vessel flattened area is equal to the area of the chamber diaphragm, the optimal amount of pressure against vessel has been applied. Various embodiments utilize different mechanisms for limiting the extent that the vessel and chamber are pressed together. This is required as beyond a limited amount of external pressure against the deformable vessel, the pressure inside the vessel will increase, and the measured pressure from the chamber will be an inaccurate measurement of the pressure inside the vessel in its resting condition. One such mechanism is the use of a spherical cup to receive a ball shaped vessel, which when the ball is seated, indicates to the user to stop pressing further, for accurate measurement. Other mechanisms utilize design configurations that make it possible to visually verify that the deformed vessel area and chamber diaphragm areas are the same.

Embodiments are disclosed for devices utilizing this method which include ball pressure measurement devices and intraocular pressure measurement.

### BRIEF DESCRIPTION OF THE DRAWINGS

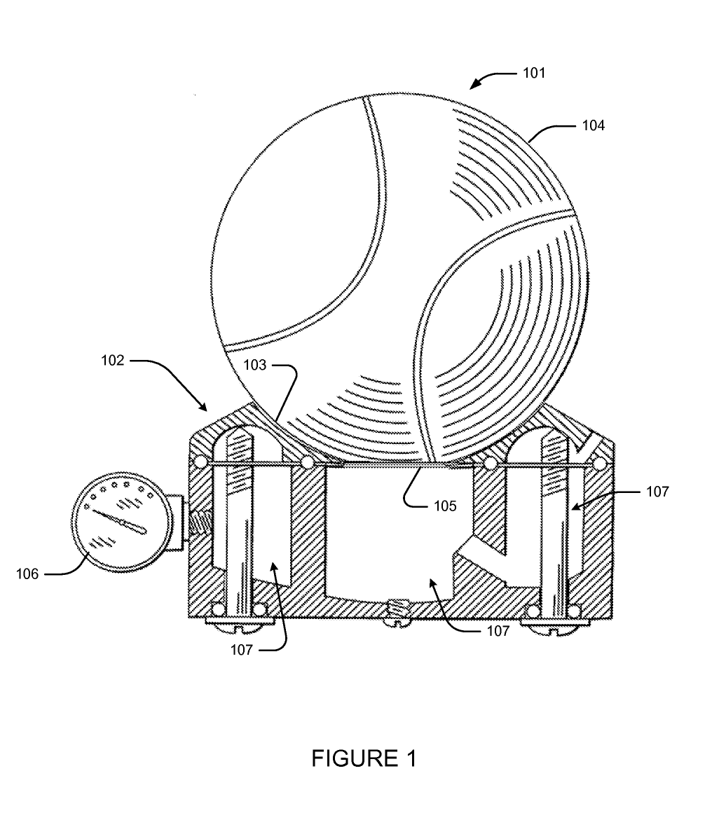

Shown in FIG. 1 is a cross section view of an embodiment for measuring pressure in tennis balls.

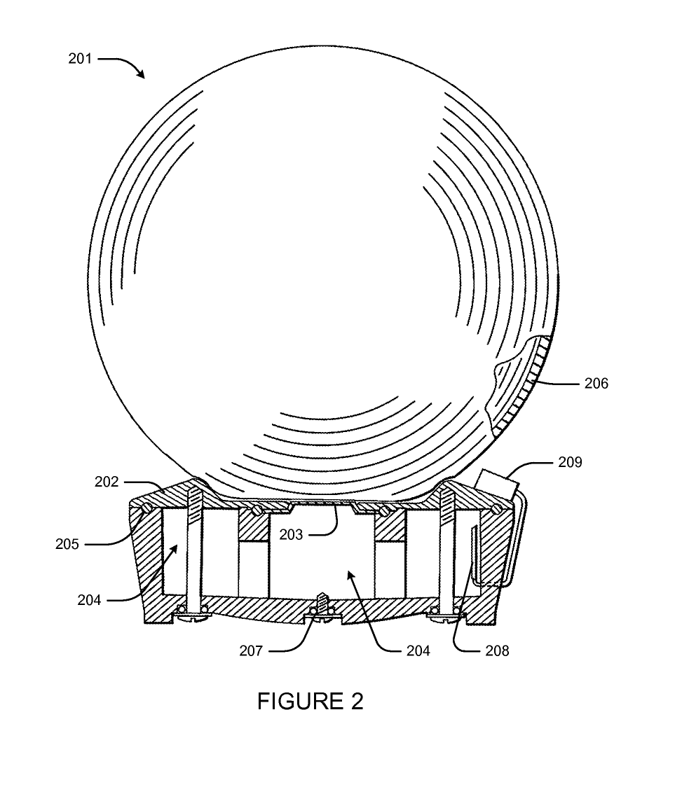

Shown in FIG. 2 is a cross section view of an embodiment device for measuring pressure in larger balls with elastic outer walls such as basketballs.

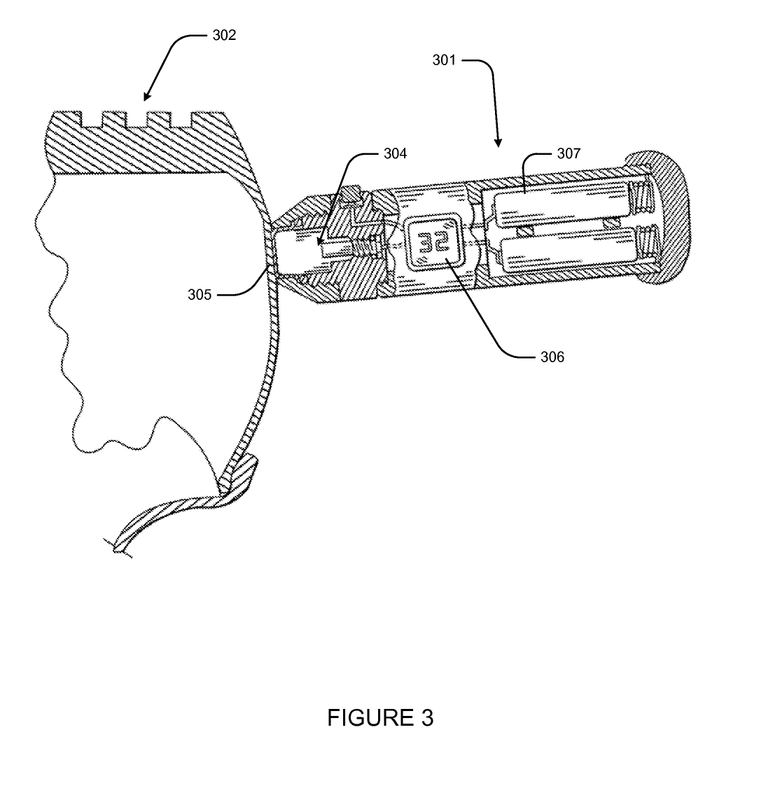

Shown in FIG. 3 is cross section view of an embodiment for measuring tire pressure against the wall of the tire.

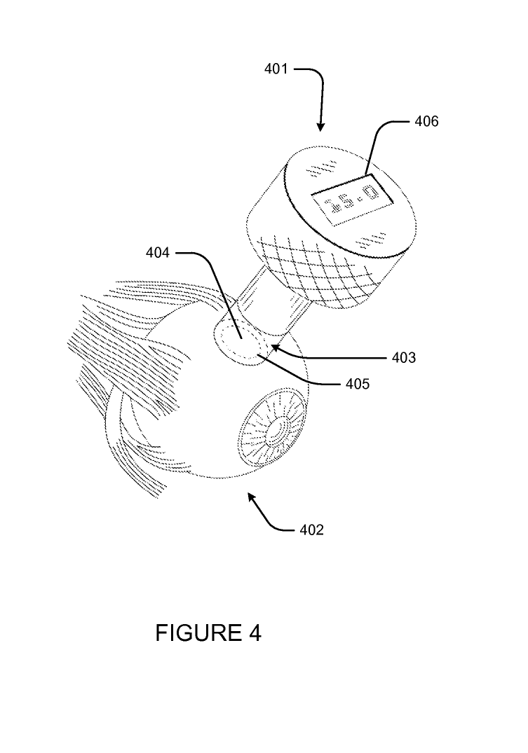

Shown in FIG. 4 is a perspective view of an embodiment for measuring intraocular pressure.

### DETAILED DESCRIPTION OF THE INVENTION

In certain embodiments as shown in FIG. 1, the sealed deformable vessel may be a ball such as a tennis ball 101. The pressure transfer and measurement apparatus 102 is situated underneath the ball, which is pressed into the upper spherical cupped receiving surface 103. The shape of the solid receiving portion of the apparatus 103 and the diameter of the deformable diaphragm 105 are determined according to the deformability or elastic properties of the outside wall of the vessel 104. As the deformable pressurized ball in pressed into the device against the diaphragm, the air pressure within the ball is transferred across the flattening diaphragm 105 to the incompressible fluid in the chamber 107. The rising pressure inside the vessel is measured in certain embodiments by various sensors or gauges such as a mechanical dial gauge 106 as shown in FIG. 1. The apparatus is designed with an optimized volume of incompressible fluid which is contiguous throughout the device which contains structural components to improve the strength of the device with orifices to fill the chamber and distribute the fluid throughout the chamber 107.

In certain embodiments designed for a lower pressure ball with a deformable wall 206, such as a basketball 201, the device platform 202 may be adjusted in size to contain a larger deformed area than a higher pressure device range. In such embodiments, the incompressible fluid pressure transfer chamber diaphragm 203 is for certain embodiments a smaller fraction of the deformed ball area than in the higher pressure range adjusted device. Also shown in this alternate embodiment are contiguous portions of the incompressible fluid chamber 204, O-rings used for sealing the assembly 205, and a fill hole 207.

In various embodiments, many types of pressure transducer/sensors may be used for the incompressible fluid chamber. In FIG. 2, a piezoelectrical or other electrical sensor 208 and a digital readout 209 are shown.

In certain embodiments, the apparatus 301 may be held in user's hand and plunged into the deformable wall of a vessel such as a pressurized tire 302 as shown in FIG. 3. In FIG. 3 the incompressible fluid chamber 304 is shown with the chamber diaphragm 305 pressed into the wall 305 of the tire 301. When the deformable tire wall 305 is flattened across the chamber window, the gauge readout 306 shows the transferred and sensed pressure on the digital display 306. In this and other various embodiments, when a steady maximum pressure is reached during measurement, an audible beep may sound as notification. The gauge sensor and display electronics are powered in certain embodiments by batteries such as those shown 307.

In certain embodiments, the device 401 may be used to measure intraocular pressure of the eye 402. As shown in FIG. 4, in various intraocular pressure measurement embodiments, the device 401 transfer chamber 403 and deformable diaphragm 404 are pressed by hand into the eyeball 402, either on directly onto the eye as shown (with application of anesthetic) or onto the eyelid. In alternate embodiments, the incompressible fluid chamber 403 is transparent, and when a set diameter 405 of the eyeball (or eyelid) is deformed into a flattened condition and a steady pressure measurement is reached, the readout can be shown on the gauge 406 and reported by the user as needed.

In certain embodiments the device is configured as a drive-on platform, wherein a vehicle tire is positioned such as to press onto the chamber diaphragm. The elasticity and deformation properties of the diaphragm and tire are utilized to calibrate the transferred pressure sensor. In certain embodiments, the device is configured to measure blood pressure by pressing the device onto tissue with arterial pressure points near the surface.

In various embodiments, the chamber incompressible fluid may be water, brake fluid, or other suitable hydraulic fluid. In various embodiments the rigid body of the chamber is configured with structural elements such as those seen in FIGS. 1 and 2, and may be formed as a molded plastic, resin, forged metal, or other appropriately selected material for the application.

In various embodiments, the chamber diaphragm is configured with an embedded reinforcing mesh to resist elastic bulges from forming in the diaphragm.

It will be understood that the particular embodiments described in detail herein are illustrative of the invention and that many other embodiments are applicable. The principal features highlighted herein may be employed in many embodiments within the scope of the claim.

I claim:

1. A method for measuring a gas pressure inside a pressurized deformable vessel comprising:
pressing the pressurized deformable vessel onto an apparatus comprising: a rigid chamber containing an incompressible fluid, a deformable diaphragm covering a window in the chamber, and a pressure sensor;
reading a displayed pressure value from a readout of the pressure sensor;
whereby the pressure within the interior of the pressurized vessel is transferred into the device chamber and measured by the pressure sensor.

2. A method as in claim 1 wherein the vessel is pressed onto the apparatus until a portion of the vessel wall is flattened against the diaphragm of substantially the same area as the diaphragm window.

3. A method as in claim 1 wherein the vessel is pressed onto the apparatus until it rests against a spherically shaped concave receptacle.

4. A method as in claim 1 wherein the vessel is a tennis ball.

5. A method as in claim 1 wherein the device is pressed onto the deformable vessel.

6. A method as in claim 1 wherein the device is pressed onto the deformable vessel and wherein the deformable vessel is a human eye.

7. A method as in claim 1 wherein the device is pressed onto the deformable vessel and wherein the deformable vessel is an inflatable tire.

8. A device for measuring gas pressure inside a pressurized deformable vessel comprising:
a rigid chamber filled with an incompressible fluid;
a deformable diaphragm configured as a barrier between the chamber interior and the chamber exterior;
a receiving portion of the chamber with the deformable diaphragm configured to receive the pressurized deformable vessel;
a pressure sensor for measuring the pressure of the incompressible fluid;
a readout showing the sensed pressure;
whereby when the vessel is pressed onto the receiving portion of the device, the pressure in the vessel is transferred to the incompressible fluid, measured by the sensor, and communicated by the readout.

9. A device as in claim 8 wherein said pressure sensor is calibrated according to an elasticity characteristic of said deformable vessel exterior wall.

10. A device as in claim 8 wherein said receiving portion of the chamber is concave spherically shaped to receive a ball shaped vessel.

11. A device as in claim 8 wherein said pressure sensor is calibrated such that it matches a resting gas pressure inside the deformable vessel when a portion of the vessel flattened against the diaphragm is of substantially the same area as the diaphragm area.

12. A device as in claim 8 wherein the receiving portion of the chamber is sized to fit a tennis ball.

13. A device as in claim 8 wherein at least a portion of the chamber is transparent.

14. A device as in claim 8 wherein the pressure sensor is a piezoelectric sensor.

15. A device as in claim 8 also comprising:
a bleed valve for facilitating the removal of air from the chamber.

16. A device as in claim 8 also comprising:
a reinforcing mesh affixed to said diagram.

17. A means for measuring pressure comprising:
a means for containing an incompressible fluid;
a means for transferring pressure from within a pressurized deformable vessel to the incompressible fluid;
a means for sensing pressure in the incompressible fluid;
a means for reading the sensed pressure in the incompressible fluid.
